(19) United States
(12) Patent Application Publication
(10) Pub. No.: US 2024/0415758 A1
(43) Pub. Date: Dec. 19, 2024

(54) COSMETIC

(71) Applicant: SHISEIDO COMPANY, LTD., Tokyo (JP)

(72) Inventor: Ayumu ENOMOTO, Tokyo (JP)

(73) Assignee: SHISEIDO COMPANY, LTD., Tokyo (JP)

(21) Appl. No.: 18/700,297

(22) PCT Filed: Oct. 28, 2022
(86) PCT No.: PCT/JP2022/040303

§ 371 Date: Apr. 11, 2024

(30) Foreign Application Priority Data
Nov. 11, 2021 (JP) 2021-184263

(51) Int. Cl. A61K 8/67; A61K 8/06; A61K 8/42; A61Q 19/08

(52) U.S. Cl. CPC A61K 8/675; A61K 8/062; A61K 8/42; A61Q 19/08; A61K 2800/48

(57) ABSTRACT

[Problem] To provide a cosmetic in which discoloration and generation of an unpleasant odor that are caused by heat or aging can be suppressed, according to the present invention.

[Solution] A cosmetic including (A) nicotinic acid or derivative thereof, (B) a compound having a carbonyl group or a compound capable of forming a carbonyl group, (C) chlorphenesin, and (D) water.

### TECHNICAL FIELD

The present invention relates to a cosmetic including nicotinic acid or derivative thereof.

### BACKGROUND ART

Nicotinic acid or derivative thereof (for example, nicotinic acid amide) is blended in cosmetics as a component effective for improving stains and wrinkles. A combination of a cosmetic including nicotinic acid amide with an additive has also been proposed (for example, Patent Literature 1).

Saccharides are components often used in cosmetics for the purpose of moisturizing, skin conditioning, thickening, and the like. However, if nicotinic acid or derivative thereof is used in combination with a saccharide, discoloration may be caused by heat or aging.

As a means for suppression of the discoloration, combination use with an inorganic salt such as sodium pyrosulfite has been proposed, but sodium pyrosulfite may generate an unpleasant odor, and therefore in some cases, cannot be blended in an amount sufficient for suppressing discoloration.

### CITATION LIST

### Patent Literature

Patent Literature 1: WO 2020/158908 A

### SUMMARY OF THE INVENTION

It has been found that browning and an unpleasant odor are often caused by combination use of nicotinic acid or derivative of nicotinic acid with a sugar, another compound having a carbonyl group, or a carbonyl group-forming compound. Then, it has been surprisingly found that discoloration and generation of an unpleasant odor that are caused by heat or aging can be suppressed by combining chlorphenesin with the above-described combination. The present invention is based on these findings.

According to the present invention, the following invention is provided.
[1] A cosmetic including:
   (A) nicotinic acid or a derivative of nicotinic acid;
   (B) a compound having a carbonyl group or a compound capable of forming a carbonyl group;
   (C) chlorphenesin; and
   (D) water.
[2] The cosmetic according to [1], wherein the component (A) is nicotinic acid amide.
[3] The cosmetic according to [1] or [2], wherein the component (B) is selected from the group consisting of monosaccharides, oligosaccharides, polysaccharides, glycosides, and sugar alcohols.
[4] The cosmetic according to any one of [1] to [3], wherein the component (B) has a thickening action.
[5] The cosmetic according to any one of [1] to [4], wherein the component (B) is selected from the group consisting of succinoglycan, xanthan gum, cellulose, guar gum, starch, agar, and derivatives thereof.
[6] The cosmetic according to any one of [1] to [5], wherein an amount of the component (A) blended is 0.1 to 10 mass % with respect to a total amount of the cosmetic.
[7] The cosmetic according to any one of [1] to [6], wherein an amount of the component (B) blended is 0.1 to 5 mass % with respect to the total amount of the cosmetic.
[8] The cosmetic according to any one of [1] to [7], wherein an amount of the component (C) blended is 0.05 to 3 mass % with respect to the total amount of the cosmetic.
[9] The cosmetic according to any one of [1] to [8], being an oil-in-water emulsion cosmetic.

According to the present invention, it is possible to suppress discoloration and generation of an unpleasant odor that are caused by a reaction between nicotinic acid or derivative of nicotinic acid and a compound having a carbonyl group or a compound capable of forming a carbonyl group over time or under a heating condition.

### BEST MODE FOR CARRYING OUT THE INVENTION

The present invention relates to a cosmetic including (A) nicotinic acid or derivative of nicotinic acid, (B) a compound having a carbonyl group or a compound capable of forming a carbonyl group, (C) chlorphenesin, and (D) water.

### (A) Nicotinic Acid or Derivative Thereof

The cosmetic according to the present invention includes (A) nicotinic acid or derivative thereof (hereinafter, sometimes referred to as a component (A), and the same applies to other components).

Examples of the derivative of nicotinic acid include nicotinic acid amide, benzyl nicotinate, tocopherol nicotinate, and nicotinic acid β-butoxy ester, and among these, nicotinic acid amide is particularly preferable. Here, nicotinic acid amide is also referred to as nicotinamide or niacinamide, is a derivative of nicotinic acid, and is an amide compound of nicotinic acid (vitamin B3/niacin). Nicotinic acid amide is a water-soluble vitamin and a substance known as one of vitamin B complex, and may be extracted from a natural product (rice bran or the like) or can be synthesized with a known method. For example, those listed in the Japanese Pharmacopoeia Eighteenth Edition can be used.

One or more kinds of the component (A) can be blended. The amount of the component (A) blended is preferably 0.1 to 10 mass %, and more preferably 0.5 to 8 mass % with respect to the total amount of the cosmetic.

### (B) Compound Having Carbonyl Group or Compound Capable of Forming Carbonyl Group

The cosmetic according to the present invention includes (B) a compound having a carbonyl group or a compound capable of forming a carbonyl group.

It is generally known that an amino group of an amino acid, a protein, or an amine reacts with a carbonyl group of a reducing sugar or the like to cause a browning phenomenon. This reaction is also called a Maillard reaction, and it is said that this reaction generates a brown pigment or an odor substance.

The component (B) is a component that can react with the component (A) having an amino group to cause a Maillard reaction. Examples of the component (B) include saccharides and polyalkylene glycol derivatives, and saccharides are particularly preferable because an effect of the present invention can be further exhibited in a case where the component (B) is a saccharide.

The saccharides are not particularly limited, and examples thereof include monosaccharides, oligosaccharides, polysaccharides, glycosides, and sugar alcohols. In the present invention, the oligosaccharides refer to those obtained by dehydration condensation of 2 to 10 molecules of monosaccharides, and the polysaccharides refer to those obtained by dehydration condensation of 11 or more molecules of monosaccharides.

Examples of the monosaccharides include tetroses such as erythrulose, erythrose, and threose, pentoses such as arabinose, xylose, lyxose, ribose, xylulose, and ribulose, hexoses such as galactose, glucose, talose, mannose, sorbose, tagatose, psicose, and fructose, branched saccharides such as apiose and hamamelose, amino sugars such as glucosamine and galactosamine, and derivatives of these monosaccharides.

Examples of the oligosaccharides include homooligosaccharides containing only one kind of the above-described monosaccharide, and heterooligosaccharides containing two or more kinds of the above-described monosaccharides. Examples of the homooligosaccharides include xylooligosaccharides such as xylobiose, xylotriose, xylotetraose, and xylopentaose, galactooligosaccharides such as agarobiose and carrabiose, glucooligosaccharides such as maltose, maltotriose, maltotetraose, maltopentaose, isomaltose, sophorose, cellobiose, cellotriose, cellotetraose, cellopentaose, trehalose, neotrehalose, and isotrehalose, mannooligosaccharides, and fructooligosaccharides such as inulobiose, inulotriose, inulotetraose, and inulopentaose. Examples of the heterooligosaccharides include vicianose, isoprimeverose, sambubiose, primeverose, lycotetraose, solabiose, melibiose, manninotriose, lactose, lycobiose, lycotriose, epicellobiose, sucrose, turanose, maltulose, isokestose, erlose, kestose, gentianose, lactulose, epigentibiose, isolychnose, umbelliferose, sesamose, raffinose, lychnose, robinobiose, silanobiose, rutinose, chacotriose, solatriose, and α-glucan oligosaccharide (glucose oligomer having a polymerization degree of 2 to 10). Among the oligosaccharides, disaccharides are preferable.

Examples of the polysaccharides include polysaccharides such as xanthan gum, cellulose, guar gum, starch, pullulan, dextran, fructan, mannan, agar, carrageenan, chitin, chitosan, pectin, alginic acid, starch, glycogen, and hyaluronic acid. The polysaccharides also include derivatives in which the above-described saccharides are substituted with a methyl group, an ethyl group, a hydroxyethyl group, a hydroxypropyl group, an acetyl group, a stearoxy group, glycerol, propylene glycol, or the like. These substituents can be used singly or in combination of two or more kinds thereof for substitution. Specific examples of the polysaccharides having a substituent include hydroxyethylcellulose, hydroxyethyl ethylcellulose, hydroxyethyl guar gum, cellulose gum, hydroxyethyl starch, methylcellulose, methyl guar gum, methyl starch, ethylcellulose, ethyl guar gum, ethyl starch, hydroxypropylcellulose, hydroxypropyl guar gum, hydroxypropyl starch, hydroxyethyl methylcellulose, hydroxyethyl methyl guar gum, hydroxyethyl methyl starch, hydroxypropyl methylcellulose, hydroxypropyl methyl guar gum, hydroxypropyl methyl starch, hydroxypropyl methylcellulose stearoxy ether, acetylated hyaluronic acid, propylene glycol alginate, and propylene glycol hyaluronate.

Examples of the glycosides include arbutin, ascorbic acid glucoside, glucosylceramide, anthocyanin, rutin, hesperidin, and daidzin.

Examples of the sugar alcohols include xylitol, trehalose, maltitol, mannitol, sorbitol, erythritol, arabitol, ribitol, galactitol, glucitol, and erythritol.

In an embodiment of the present invention, the component (B) preferably has a thickening action for improvement in formulation stability, and the component (B) is more preferably selected from the group consisting of succinoglycan, xanthan gum, cellulose, guar gum, starch, agar, and derivatives thereof.

In another embodiment of the present invention, the component (B) is preferably selected from the group consisting of monosaccharides, oligosaccharides, and glycosides, and the component (B) is more preferably a monosaccharide or a disaccharide, and still more preferably fructose, glucose, mannose, or maltose.

One or more kinds of the component (B) can be blended. The amount of the component (B) blended is preferably 0.1 to 5 mass %, and more preferably 0.3 to 3 mass % with respect to the total amount of the cosmetic.

### (C) Chlorphenesin

The cosmetic according to the present invention includes (C) chlorphenesin, that is, 3-(4-chlorophenoxy)-1,2-propanediol.

Chlorphenesin is known to have antibacterial activity, and is generally blended in cosmetics as a preservative for suppression of growth of microorganisms. However, in the present invention, chlorphenesin exhibits a function different from that described above. As described above, the combination of the component (A) and the component (B) may cause discoloration and generation of an unpleasant odor. The present inventor surprisingly has found that the discoloration and the generation of an unpleasant odor are suppressed by further combining chlorphenesin with the combination of the component (A) and the component (B). In the present invention, chlorphenesin is considered to function as a Maillard reaction inhibitor.

The amount of the component (C) blended is preferably 0.05 to 3 mass %, and more preferably 0.1 to 1 mass % with respect to the total amount of the cosmetic.

### (D) Water

The cosmetic according to the present invention includes (D) water. As the water, water used in cosmetics, quasi-drugs, and the like can be used, and for example, purified water, ion-exchanged water, tap water, or the like can be used.

The amount of the water blended is preferably 5 to 90 mass %, and more preferably 10 to 80 mass % with respect to the total amount of the cosmetic according to the present invention.

In the cosmetic according to the present invention, in addition to the above-described components, a component usually used in cosmetics, such as an oily component, an ultraviolet absorber, a surfactant, a moisturizer, a thickener, a polyhydric alcohol, a powder component, an antioxidant, or a preservative, can be appropriately blended as necessary.

The form of the cosmetic according to the present invention is not particularly limited, but is preferably an oil-in-water emulsion cosmetic. Specific use is possible as a skin care cosmetic such as a lotion, a gel, a milky lotion, a cream, or a pack, a makeup cosmetic such as a makeup base or a foundation, a base cosmetic, or a sunscreen cosmetic.

The cosmetic of the present invention can be produced in accordance with a conventional method. In a case where the cosmetic according to the present invention is an emulsion cosmetic, the method of emulsification is not particularly limited.

### EXAMPLES

The present invention will be described specifically with reference to the following Examples, but the present invention is not limited to these Examples. The content is shown in mass % with respect to the total amount unless otherwise specified.

### Examples 1 to 4 and Comparative Examples 1 to 5

Cosmetics of Examples 1 to 4 and Comparative Examples 1 to 5 were prepared at combinations shown in Table 1.

TABLE 1

| | | Example | | | | Comparative Example | | | | |
|---|---|---|---|---|---|---|---|---|---|---|
| | | 1 | 2 | 3 | 4 | 1 | 2 | 3 | 4 | 5 |
| Composition | Nicotinic acid amide | 5 | 5 | 5 | 5 | 5 | 5 | 5 | 5 | 5 |
| | Succinoglycan | 1 | — | — | — | 1 | 1 | — | — | — |
| | Xanthan gum | — | 1 | — | — | — | — | 1 | — | — |
| | Hydroxypropyl | — | — | 1 | — | — | — | — | 1 | — |
| | methylcellulose | | | | | | | | | |
| | stearoxy ether | | | | | | | | | |
| | Agar | — | — | — | 1 | — | — | — | — | 1 |
| | Chlorphenesin | 0.2 | 0.2 | 0.2 | 0.2 | — | — | — | — | — |
| | Sodium pyrosulfite | — | — | — | — | 0.2 | — | — | — | — |
| | BG (butylene glycol) | 5 | 5 | 5 | 5 | 5 | 5 | 5 | 5 | 5 |
| | Ethanol | 4 | 4 | 4 | 4 | 4 | 4 | 4 | 4 | 4 |
| | Water and another | Balance | Balance | Balance | Balance | Balance | Balance | Balance | Balance | Balance |
| | component | | | | | | | | | |
| | Total | 100 | 100 | 100 | 100 | 100 | 100 | 100 | 100 | 100 |
| Evaluation | Odor | A | A | A | A | C | B | B | B | B |
| | Discoloration | A | A | A | A | A | C | C | C | C |

### [Odor Evaluation]

The samples after keeping the temperature were subjected to sensory evaluation by an expert panelist, and judged in accordance with the following criteria. The obtained results are as shown in Table 1.
A: Few unpleasant odor was felt.
B: An unpleasant odor was felt.
C: An unpleasant odor was strongly felt.

### [Discoloration Evaluation]

Each of the cosmetics of Examples and Comparative Examples was put in a 50 ml sample tube, and kept warm in a thermostatic bath at 70° C. for 3 days. The sample after keeping the temperature was colorimetrically measured using a color difference meter (“Spectrophotometer CM-2002”, KONICA MINOLTA, INC.) to obtain L, a, and b values, and the color difference ΔE was calculated between the obtained colorimetric values and the values measured using ion-exchanged water, and evaluated in accordance with the following criteria.
A: ΔE is less than 1.0.
B: ΔE is 1.0 or more and less than 5.0.
C: ΔE is 5.0 or more.

### Formulation Examples 1 to 7

Formulation Examples 1 to 7 of the cosmetic of the present invention are shown in the following Tables 2 to 7.

TABLE 2

| | Formulation | Formulation |
|---|---|---|
| | Example 1 | Example 2 |
| Water | Balance | Balance |
| Dynamite glycerol | 2 | 2 |
| BG | 5 | 5 |
| Ethanol | 4 | 4 |
| Phenoxyethanol | 0.5 | — |
| Methylparaben | — | 0.1 |
| Chlorphenesin | 0.2 | 0.2 |
| Hydroxypropyl methylcellulose | 0.1 | 0.1 |
| stearoxy ether | | |
| Succinoglycan | 0.15 | 0.15 |
| (Dimethylacrylamide/sodium | 0.25 | 0.25 |
| acryloyldimethyltaurate) | | |
| crosspolymer | | |
| PEG-100 hydrogenated | 1.5 | 1.5 |
| castor oil | | |
| Dimethicone | 13 | 13 |
| Isostearic acid | 1 | 1 |
| Distearyldimonium chloride | 0.1 | 0.1 |
| Hydrophobized zinc oxide | 12 | 12 |
| Hydrophobized titanium oxide | 1 | 1 |
| Ethylhexyl triazone | 0.5 | 0.5 |
| Hexyl diethylamino | 0.5 | 0.5 |
| hydroxybenzoyl benzoate | | |
| Bis-ethylhexyloxyphenol | 2 | 2 |
| methoxyphenyl triazine | | |
| Ethylhexyl methoxycinnamate | 7 | 7 |
| Hydrogenated palm oil | 1 | 1 |
| Isopropyl myristate | 2 | 2 |
| Dextrin palmitate/ethylhexanoate | 0.5 | 0.5 |
| BHT (dibutylhydroxytoluene) | 0.05 | 0.05 |
| Sodium pyrosulfite | 0.001 | 0.001 |
| Tocopherol | 0.05 | 0.05 |
| Nicotinic acid amide | 5 | 5 |
| Total | 100 | 100 |

TABLE 3

| | Formulation |
|---|---|
| | Example 3 |
| Water | Balance |
| Nicotinic acid amide | 5 |
| Glycerin | 2.5 |
| DPG (dipropylene glycol) | 5 |
| PEG-60 glyceryl isostearate | 0.3 |
| PEG-10 dimethicone | 0.3 |
| TEA (triethanolamine) | 0.3 |
| Behenyl alcohol | 1 |
| Batyl alcohol | 1.5 |
| Behenic acid | 0.6 |
| Pentaerythritol tetra(2- | 2 |
| ethylhexanoate) | |
| Polypropylene glycol (17) | 1 |
| Cyclopentasiloxane | 4 |
| Dimethicone (6 cst) | 1 |
| Ethylhexyl methoxycinnamate | 7.5 |
| Bis-ethylhexyloxyphenol | 2 |
| methoxyphenyl triazine | |
| Xanthan gum | 0.2 |
| Phenoxyethanol | 0.35 |
| Chlorphenesin | 0.2 |
| Thiotaurine | 0.1 |
| Aminoethanesulfinic acid | 0.1 |
| Rosa multiflora | 0.1 |
| fruit extract | |
| Rosa centifolia | 0.1 |
| flower extract | |
| Titanium oxide | 0.05 |
| Mica | 0.05 |
| Chelating agent | Appropriate amount |
| pH adjuster | Appropriate amount |
| Total | 100 |

TABLE 4

| | Formulation |
|---|---|
| | Example 4 |
| Water | Balance |
| Nicotinic acid amide | 5 |
| Glycerin | 4 |
| BG | 7 |
| PEG/PPG-9/2 dimethyl ether | 5 |
| Polyethylene glycol 300 | 1 |
| Succinoglycan | 0.3 |
| (Dimethylacrylamide/sodium | 0.3 |
| acryloyldimethyltaurate) | |
| crosspolymer | |
| Sucrose fatty acid ester | 3 |
| PEG-60 hydrogenated castor oil | 2 |
| Diisopropyl sebacate | 2 |
| Nonvolatile dimethicone | 2 |
| Cyclomethicone | 12 |
| Triethylhexanoin | 5 |
| Isostearic acid | 1 |
| Sorbitan sesquiisostearate | 0.5 |
| Hydrophobized zinc | 10 |
| oxide fine particle | |
| Ethylhexyl methoxycinnamate | 10 |
| Hexyl diethylamino | 1 |
| hydroxybenzoyl benzoate | |
| Bis-ethylhexyloxyphenol | 1 |
| methoxyphenyl triazine | |
| Urethane resin | 3 |
| spherical powder | |
| Chlorphenesin | 0.2 |
| pH adjuster | Appropriate amount |
| Chelating agent | Appropriate amount |
| Total | 100 |

TABLE 5

| | Formulation |
|---|---|
| | Example 5 |
| Water | Balance |
| Nicotinic acid amide | 5 |
| Ethylhexyl methoxycinnamate | 7.5 |
| Stearic acid | 10 |
| Potassium hydroxide | 0.5 |
| Xanthan gum | 0.2 |
| Chlorphenesin | 0.2 |
| Total | 100 |

TABLE 6

| | Formulation |
|---|---|
| | Example 6 |
| Water | Balance |
| Nicotinic acid amide | 5 |
| Diisopropyl sebacate | 8 |
| Bis-ethylhexyloxyphenol | 1 |
| methoxyphenyl triazine | |
| Isopentyl trimethoxycinnamate | 1 |
| trisiloxane | |
| Hydrophobized zinc oxide | 5 |
| Succinoglycan | 1 |
| (Dimethylacrylamide/sodium | 0.3 |
| acryloyldimethyltaurate) | |
| crosspolymer | |
| Hydroxypropyl methylcellulose | 0.2 |
| stearoxy ether | |
| Chlorphenesin | 0.2 |
| BG | 4 |
| Ethanol | 4 |
| Total | 100 |

TABLE 7

| | Formulation |
|---|---|
| | Example 7 |
| Water | Balance |
| Nicotinic acid amide | 5 |
| Ethanol | 3 |
| Cellulose gum | 0.1 |
| Glycerin | 2 |
| Isododecane | 5 |
| Isopropyl myristate | 5 |
| Diisopropyl sebacate | 10 |
| PBG/PPG-9/1 copolymer | 1 |
| Dimethicone 1.5 cs | 10 |
| Dimethicone (6 cs) | 5 |
| Trimethylsiloxysilicate | 1 |
| Dextrin palmitate | 1 |
| Ethylhexyl methoxycinnamate | 5 |
| Hexyl diethylamino | 1 |
| hydroxybenzoyl benzoate | |
| Bis-ethylhexyloxyphenol | 1 |
| methoxyphenyl triazine | |
| Ethylhexyl salicylate | 3 |
| Hydrophobized titanium | 5 |
| oxide fine particle | |
| Hydrophobized zinc | 10 |
| oxide fine particle | |
| PEG-9 polydimethylsiloxyethyl | 1 |
| dimethicone | |
| Cetyl PEG/PPG-10/1 | 1 |
| dimethicone | |
| Dimethyldistearyl | 0.2 |
| ammonium hectorite | |
| Chlorphenesin | 0.1 |
| Isostearic acid | 0.1 |
| Chelating agent | Appropriate amount |
| Thiotaurine | 0.1 |
| Aminoethanesulfinic | 0.1 |
| acid | |
| Rosa multiflora | 0.1 |
| fruit extract | |
| Rosa centifolia | 0.1 |
| flower extract | |
| Titanium oxide | 0.05 |
| Mica | 0.05 |
| Total | 100 |

1. A cosmetic comprising:
(A) nicotinic acid or a derivative thereof;
(B) a compound having a carbonyl group or a compound capable of forming a carbonyl group;
(C) chlorphenesin; and
(D) water.

2. The cosmetic according to claim 1, wherein the component (A) is nicotinic acid amide.

3. The cosmetic according to claim 1, wherein the component (B) is selected from the group consisting of monosaccharides, oligosaccharides, polysaccharides, glycosides, and sugar alcohols.

4. The cosmetic according to claim 1, wherein the component (B) has a thickening action.

5. The cosmetic according to claim 1, wherein the component (B) is selected from the group consisting of succinoglycan, xanthan gum, cellulose, guar gum, starch, agar, and derivatives thereof.

6. The cosmetic according to claim 1, wherein an amount of the component (A) blended is 0.1 to 10 mass % with respect to a total amount of the cosmetic.

7. The cosmetic according to claim 1, wherein an amount of the component (B) blended is 0.1 to 5 mass % with respect to the total amount of the cosmetic.

8. The cosmetic according to claim 1, wherein an amount of the component (C) blended is 0.05 to 3 mass % with respect to the total amount of the cosmetic.

9. The cosmetic according to claim 1, being an oil-in-water emulsion cosmetic.
